(19) United States
(12) Patent Application Publication
(10) Pub. No.: US 2025/0157231 A1
(43) Pub. Date: May 15, 2025

(54) IMAGE STANDARDIZATION METHOD AND OBSERVATION APPARATUS

(71) Applicant: SCREEN Holdings Co., Ltd., Kyoto (JP)

(72) Inventors: Ryo HASEBE, Kyoto-shi, Kyoto (JP); Yasushi KUROMI, Kyoto-shi, Kyoto (JP)

(21) Appl. No.: 18/838,336

(22) PCT Filed: Dec. 20, 2022
(86) PCT No.: PCT/JP2022/046875

§ 371 Date: Aug. 14, 2024

(30) Foreign Application Priority Data
Mar. 18, 2022 (JP) 2022-043442

(51) Int. Cl. G06V 20/69; G06V 10/25; G06V 10/60

(52) U.S. Cl. CPC G06V 20/695; G06V 10/25; G06V 10/60; G06T 2207/10101

(57) ABSTRACT

According to this image standardization method, first, a region corresponding to a whole or a part of a biological sample in an image is extracted as an observation target region. Subsequently, an aggregate value of intensity values is calculated for each of multiple observation target regions. After that, intensity values of the multiple observation target regions are adjusted so that a difference in the aggregate value becomes smaller. This reduces variation in brightness among the multiple observation target regions. This can reduce a difference in an intensity value of the observation target region for the image of the biological sample including multiple cells. Consequently, multiple biological samples can be fairly observed and evaluated.

### TECHNICAL FIELD

The present invention relates to a technology for standardizing an intensity value of an image of a biological sample including multiple cells.

### BACKGROUND ART

There is conventionally known an observation apparatus that takes an image of a biological sample with multiple cells by optical coherence tomography (OCT), and observes the biological sample on the basis of the obtained tomographic image. Such a conventional observation apparatus is described in, for example, PTL 1. With the use of this type of observation apparatus, it is possible to noninvasively observe a three-dimensional structure of a biological sample.

### CITATION LIST

### Patent Literature

PTL 1: Japanese Patent Application Laid-Open No. 2018-105683

### SUMMARY OF INVENTION

### Technical Problem

In the above-mentioned observation apparatus, in performing optical coherence tomography, near infrared light is applied to a biological sample that, together with a culture medium, is held by a sample holder such as a well plate. At that time, the near infrared light passes through the sample holder and the culture medium before being applied to the biological sample. Thus, an amount of the near infrared light applied to the biological sample is affected by a material, a thickness, a shape, and a surface coating of the sample holder, an amount of the culture medium, and the like. Hence, as those factors vary, an intensity of a tomographic image to be obtained varies.

Further, biological samples including multiple cells, such as spheroids or organoids, have different shapes and sizes, respectively. Thus, even though environmental factors such as a sample holder and a culture medium are constant, there occurs variation in an amount of near infrared light reaching the inside of each biological sample due to differences in a shape and a size among the biological samples. As a result, there occurs variation in an intensity value among tomographic images in some cases.

The present invention has been made in view of the above-described situation, and it is an object to provide a technology that enables reduction of a difference in an intensity value of an observation target region for an image of a biological sample including multiple cells.

### Solution to Problem

In order to solve the above-described problem, the first invention of the present application is directed to an image standardization method for standardizing intensity values of images of a biological sample including multiple cells, including the steps of: a) extracting a region corresponding to a whole or a part of the biological sample in each of the images, as an observation target region; b) calculating an aggregate value of intensity values for each of the multiple observation target regions; and c) adjusting intensity values of the multiple observation target regions so that a difference in the aggregate value becomes smaller.

The second invention of the present application is directed to the image standardization method according to the first invention, wherein the aggregate value is an average value of multiple intensity values included in the observation target region.

The third invention of the present application is directed to the image standardization method according to the first invention, wherein the aggregate value is a median value of multiple intensity values included in the observation target region.

The fourth invention of the present application is directed to the image standardization method according to any of the first invention to the third invention, wherein the region corresponding to the biological sample in each of the images includes multiple regions having different intensity values, and in the step a), one of the multiple regions is extracted as the observation target region.

The fifth invention of the present application is directed to the image standardization method according to any of the first invention to the fourth invention, wherein the images are tomographic images or three-dimensional images of the biological sample that are acquired by optical coherence tomography.

The sixth invention of the present application is directed to an observation apparatus for observing a biological sample including multiple cells, including: an image acquisition unit configured to acquire images of the biological sample: a region extraction unit configured to extract a region corresponding to a whole or a part of the biological sample in each of the images, as an observation target region; an aggregate-value calculation unit configured to calculate an aggregate value of intensity values, for each of the multiple observation target regions; and an intensity-value adjustment unit configured to adjust intensity values of the multiple observation target regions so that a difference in the aggregate value becomes smaller.

The seventh invention of the present application is directed to the observation apparatus according to the sixth invention, wherein the aggregate value is an average value of multiple intensity values included in the observation target region.

The eighth invention of the present application is directed to the observation apparatus according to the sixth invention, wherein the aggregate value is a median value of multiple intensity values included in the observation target region.

The ninth invention of the present application is directed to the observation apparatus according to any of the sixth invention to the eighth invention, wherein the region corresponding to the biological sample in each of the images includes multiple regions having different intensity values, and the region extraction unit extracts one of the multiple regions as the observation target region.

The tenth invention of the present application is directed to the observation apparatus according to any of the sixth invention to the ninth invention, wherein the image acquisition unit acquires a tomographic image or a three-dimensional image of the biological sample by optical coherence tomography.

The eleventh invention of the present application is directed to the observation apparatus according to any of the sixth invention to the tenth invention, further including an evaluation-value output unit configured to output an evaluation value of the biological sample on the basis of an image having an intensity value having been adjusted by the intensity-value adjustment unit.

### Advantageous Effects of Invention

According to the first invention to the eleventh invention of the present application, variation in brightness among the multiple observation target regions is reduced. Therefore, multiple biological samples can be fairly observed and evaluated.

Especially, according to the third invention and the eighth invention of the present application, even in a case in which there is an outlier pixel having an extremely remote intensity value in an observation target region, the influence of the outlier can be reduced, and an aggregate value that reflects the overall brightness of the observation target region can be calculated.

Especially, according to the fifth invention and the tenth invention of the present application, variation in an intensity value among biological samples, which is likely to occur in optical coherence tomography, in particular, can be reduced.

### BRIEF DESCRIPTION OF DRAWINGS

FIG. 1 is a diagram showing a configuration of an observation apparatus.

FIG. 2 is a control block diagram of the observation apparatus.

FIG. 3 is a block diagram conceptually showing functions of a computer.

FIG. 4 is a flowchart showing a flow of an image-taking and evaluating process.

FIG. 5 is a view schematically showing multiple tomographic images.

FIG. 6 is a view schematically showing a result of extraction of observation target regions from tomographic images.

FIG. 7 is a view schematically showing multiple tomographic images having been subjected to standardization.

FIG. 8 is a view schematically showing an example of a tomographic image including multiple regions having different intensity values.

### DESCRIPTION OF EMBODIMENTS

Hereinafter, an embodiment of the present invention will be described with reference to the drawings.

### <1. Configuration of Observation Apparatus>

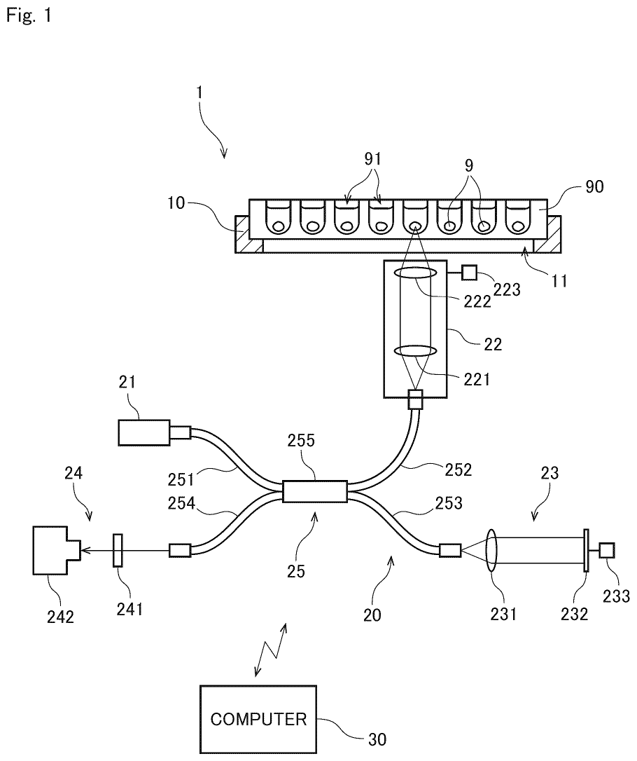

FIG. 1 is a view showing a configuration of an observation apparatus 1 according to one embodiment of the present invention. The observation apparatus 1 is an apparatus that takes an image of a biological sample 9 held in a sample holder 90 and evaluates a condition of the biological sample 9 on the basis of the acquired image. The biological sample 9 is a cell aggregate such as a spheroid or an organoid including multiple cells. The biological sample 9 may be, for example, a cell aggregate obtained from stem cells for regenerative medicine, or may be an embryo formed as a result of cleavage of a fertilized egg. Alternatively, the biological sample 9 may be a tumor tissue or the like used for screening in drug discovery.

As shown in FIG. 1, the observation apparatus 1 includes a stage 10, an imaging unit 20, and a computer 30.

The stage 10 is a support plate that supports the sample holder 90. For the sample holder 90, for example, a well plate is used. The well plate consists of multiple wells (recessed portions) 91. Each of the wells 91 has a U-shaped or V-shaped bottom. The biological sample 9, together with a culture medium, is held near the bottom of each well 91. For a material of the sample holder 90, transparent resin or glass that transmits light is used.

The stage 10 includes an opening 11 that vertically penetrates. The sample holder 90 is horizontally supported by the stage 10. Then, most of a lower surface of the sample holder 90 is placed in the opening 11. Thus, the lower surface of the sample holder 90 is exposed toward the imaging unit 20 without being covered by the stage 10.

The imaging unit 20 is a unit that takes an image of the biological sample 9 in the sample holder 90. The imaging unit 20 is placed below the sample holder 90 supported by the stage 10. The imaging unit 20 of the present embodiment is an optical coherence tomography (OCT) device capable of taking a tomographic image and a three-dimensional image of the biological sample 9.

As shown in FIG. 1, the imaging unit 20 includes a light source 21, an object optical system 22, a reference optical system 23, a detection unit 24, and an optical fiber coupler 25. The optical fiber coupler 25 includes first to fourth optical fibers 251 to 254 connected at a connecting unit 255. The light source 21, the object optical system 22, the reference optical system 23, and the detection unit 24 are connected to each other via optical paths formed by the optical fiber coupler 25.

The light source 21 includes a light emitting element such as an LED. The light source 21 emits low-coherence light including a wide range of wavelength components. In order to allow light to reach the inside of the biological sample 9 without invasively treating the biological sample 9, it is desirable that light emitted from the light source 21 is a near-infrared ray. The light source 21 is connected to the first optical fiber 251. Light emitted from the light source 21 is incident on the first optical fiber 251 and is separated into light incident on the second optical fiber 252 and light incident on the third optical fiber 253 at the connecting unit 255.

The second optical fiber 252 is connected to the object optical system 22. Light traveling from the connecting unit 255 to the second optical fiber 252 is incident on the object optical system 22. The object optical system 22 includes multiple optical components including a collimator lens 221 and an object lens 222. Light emitted from the second optical fiber 252 passes through the collimator lens 221 and the object lens 222, and is applied to the biological sample 9 in the sample holder 90. At that time, the object lens 222 causes the light to converge to the biological sample 9. Then, light reflected from the biological sample 9 (hereinafter referred to as “observation light”) passes through the object lens 222 and the collimator lens 221 and is again incident on the second optical fiber 252.

As shown in FIG. 1, the object optical system 22 is connected to a scan mechanism 223. The scan mechanism 223 slightly moves the object optical system 22 vertically and horizontally in accordance with an instruction from the computer 30. Thus, the incidence position of light on the biological sample 9 can be slightly moved vertically and horizontally.

Further, the imaging unit 20 can be moved horizontally by a movement mechanism not shown. Thus, the field of view of the imaging unit 20 can be changed among the multiple wells 91.

The third optical fiber 253 is connected to the reference optical system 23. Light travelling from the connecting unit 255 to the third optical fiber 253 is incident on the reference optical system 23. The reference optical system 23 includes a collimator lens 231 and a mirror 232. Light emitted from the third optical fiber 253 passes through the collimator lens 231 and is incident on the mirror 232. Then, light reflected from the mirror 232 (hereinafter referred to as “reference light”) passes through the collimator lens 231 and is again incident on the third optical fiber 253.

As shown in FIG. 1, the mirror 232 is connected to a retraction mechanism 233. The retraction mechanism 233 slightly moves the mirror 232 in an optical-axis direction in accordance with an instruction from the computer 30. Thus, the optical-path length of the reference light can be changed.

The fourth optical fiber 254 is connected to the detection unit 24. The observation light that is incident on the second optical fiber 252 from the object optical system 22 and the reference light that is incident on the third optical fiber 253 from the reference optical system 23 join together at the connecting unit 255, and are incident on the fourth optical fiber 254. Then, light emitted from the fourth optical fiber 254 is incident on the detection unit 24. At that time, interference is caused between the observation light and the reference light due to a phase difference therebetween. The optical spectrum of interference light at that time varies with a height of reflection position of the observation light.

The detection unit 24 includes a spectroscope 241 and a light detector 242. The interference light emitted from the fourth optical fiber 254 is dispersed into each wavelength component in the spectroscope 241, and is incident on the light detector 242. The light detector 242 detects the interference light having been dispersed, and outputs its corresponding detection signal to the computer 30.

The image acquisition unit 41 described later in the computer 30 performs Fourier transform on the detection signal provided from the light detector 242, to thereby calculate a vertical light-intensity distribution of the observation light. Further, the image acquisition unit 41 repeats the above-described calculation of light-intensity distribution while the object optical system 22 is horizontally moved by the scan mechanism 223, to thereby calculate a light-intensity distribution of the observation light at each coordinate position in a three-dimensional space. Consequently, the computer 30 can acquire a tomographic image and a three-dimensional image of the biological sample 9.

A tomographic image is formed of multiple pixels arranged on two-dimensional coordinates, and is data in which each pixel has a predetermined intensity value. A three-dimensional image is formed of multiple voxels arranged on three-dimensional coordinates and is data in which each voxel has a predetermined intensity value.

The computer 30 has a function as a control unit that controls an operation of the imaging unit 20. Further, the computer 30 has a function as an evaluation processing unit that produces a tomographic image and a three-dimensional image on the basis of a detection signal input from the imaging unit 20 and evaluates a condition of the biological sample 9 on the basis of the acquired tomographic image and three-dimensional image.

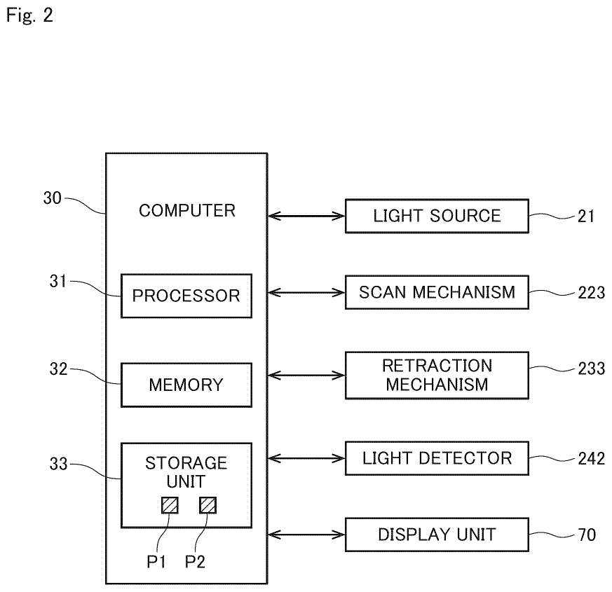

FIG. 2 is a control block diagram of the observation apparatus 1. As conceptually shown in FIG. 2, the computer 30 includes a processor 31 such as a CPU, a memory 32 such as a RAM, and a storage unit 33 such as a hard disk drive. In the storage unit 33, a control program P1 for controlling operations of respective components in the observation apparatus 1 and an evaluation program P2 for producing a tomographic image and a three-dimensional image and evaluating a condition of the biological sample 9, are stored.

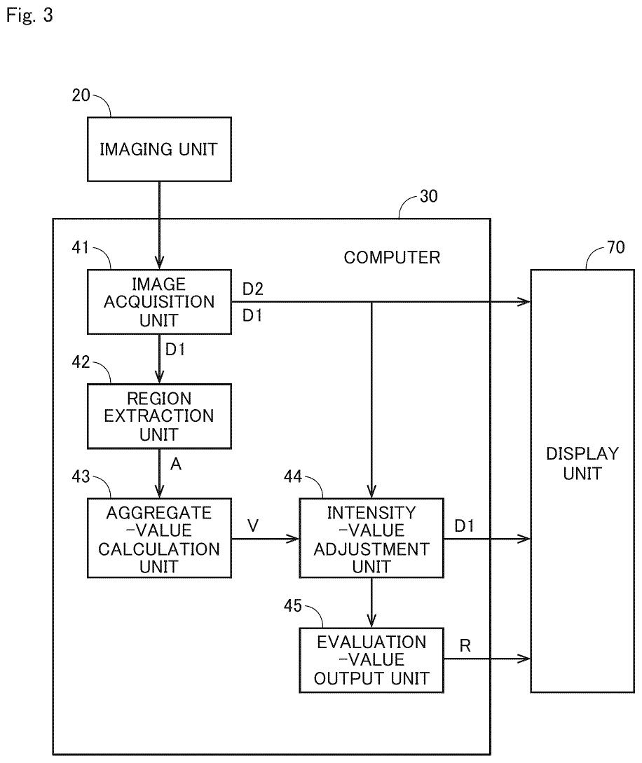

Further, as shown in FIG. 3, the computer 30 is connected to the light source 21, the scan mechanism 223, the retraction mechanism 233, and the light detector 242 that have been described above, and to a display unit 70 described later such that the computer 30 can conduct communication with each of those components. The computer 30 controls operations of the above-described respective components in accordance with the control program P1. Thus, an image-taking process of the biological sample 9 held in the sample holder 90 proceeds.

### <2. Image-Taking and Evaluating Process>

Next, an image-taking and evaluating process of the biological sample 9 in the above-described observation apparatus 1 is described.

FIG. 3 is a block diagram conceptually showing functions of the computer 30 for performing the image-taking and evaluating process. As shown in FIG. 3, the computer 30 includes an image acquisition unit 41, a region extraction unit 42, an aggregate-value calculation unit 43, an intensity-value adjustment unit 44, and an evaluation-value output unit 45. The respective functions of the image acquisition unit 41, the region extraction unit 42, the aggregate-value calculation unit 43, the intensity-value adjustment unit 44, and the evaluation-value output unit 45 are performed by an operation of the processor 31 of the computer 30 in accordance with the evaluation program P2 described above.

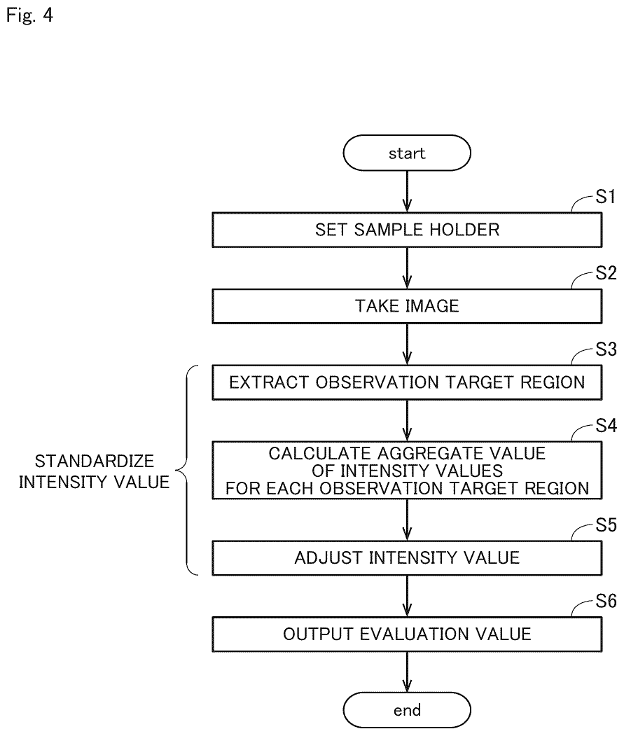

FIG. 4 is a flowchart showing a flow of the image-taking and evaluating process. In taking an image of the biological sample 9 and evaluating it in the observation apparatus 1, first, the sample holder 90 is set on the stage 10 (step S1). In the sample holder 90, the biological sample 9, together with a culture medium, is held.

Secondly, the observation apparatus 1 takes an image of the biological sample 9 with the use of the imaging unit 20 (step S2). In the present embodiment, the imaging unit 20 performs optical coherence tomography. Specifically, the light source 21 is caused to emit light, and interference light of observation light and reference light is detected for each wavelength component by the light detector 242 while the object optical system 22 is slightly moved by the scan mechanism 223. The image acquisition unit 41 of the computer 30 calculates a light-intensity distribution at each coordinate position of the biological sample 9 on the basis of a detection signal output from the light detector 242. Thus, a tomographic image D1 and a three-dimensional image D2 of the biological sample 9 are acquired.

The observation apparatus 1 acquires multiple tomographic images D1 and one three-dimensional image D2 for one biological sample 9. Further, the observation apparatus 1 repeats the process of the step S2 while changing the well 91 that is a target of image taking, to thereby acquire the tomographic images D1 and the three-dimensional images D2 of multiple biological samples 9. The acquired tomographic images D1 and three-dimensional images D2 are stored in the storage unit 33 of the computer 30. Moreover, the computer 30 displays the acquired tomographic images D1 and three-dimensional images D2 on the display unit 70.

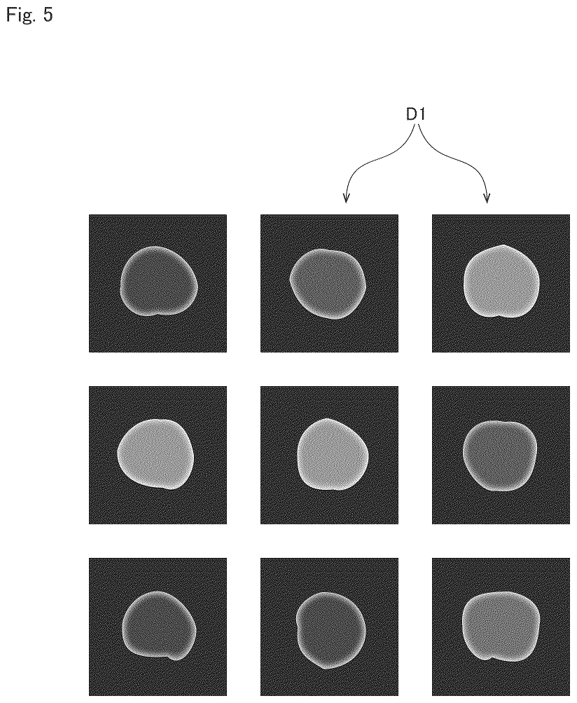

FIG. 5 is a view schematically showing the multiple tomographic images D1. When there is variation in a material, a thickness, a shape and a surface coating of the sample holder 90, an amount of a culture medium, and the like, a difference in an amount of a near infrared ray applied to the biological sample 9 is caused. In such a case, as shown in FIG. 5, the multiple tomographic images D1 acquired by optical coherence tomography include relatively bright images and relatively dark images in a mixed manner. The computer 30 performs standardization of intensity value on those multiple tomographic images D1. The standardization of intensity value is achieved by processes of steps S3 to S5 shown in FIG. 4.

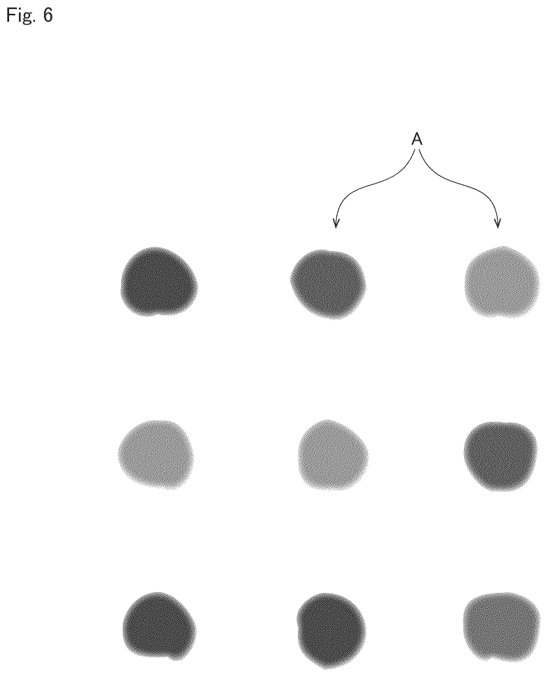

First, the region extraction unit 42 of the computer 30 extracts an observation target region A for each of the multiple tomographic images D1 (step S3). The region extraction unit 42 extracts a region corresponding to the biological sample 9 in each of the tomographic images D1, as the observation target region A. FIG. 6 is a view schematically showing a result of extraction of the observation target region A for each of the multiple tomographic images D1.

In the step S3, for example, a region having an intensity value higher than a predetermined threshold value in the tomographic image D1 is extracted as the observation target region A. Alternatively, a learning model for extracting the observation target region A from the tomographic image D1 may be created in advance with the use of deep learning. Then, the observation target region A may be extracted with the use of the learning model.

Secondly, the aggregate-value calculation unit 43 of the computer 30 calculates an aggregate value V of intensity values for each of the multiple observation target regions A extracted in the step S3 (step S4). The aggregate value V is an indicator representing overall brightness of one observation target region A. In the present embodiment, the aggregate-value calculation unit 43 uses an average value of intensity values of multiple pixels included in the observation target region A, as the aggregate value V. The aggregate-value calculation unit 43 calculates one aggregate value V for one observation target region A.

Subsequently, the intensity-value adjustment unit 44 of the computer 30 adjusts intensity values of the multiple observation target regions A on the basis of the aggregate values V calculated in the step S4 (step S5). The intensity-value adjustment unit 44 adjusts the intensity values of the observation target regions A of the respective tomographic images D1 so that a difference in the aggregate value V among the multiple observation target regions A becomes smaller. For example, for the observation target region A whose aggregate value V of intensity values is larger than a predetermined reference value, an intensity value of each pixel is lowered. Meanwhile, for the observation target region A whose aggregate value V of intensity values is smaller than the predetermined reference value, an intensity value of each pixel is increased.

Further, the intensity-value adjustment unit 44 may divide an intensity value of each pixel in the observation target region A by the aggregate value V of the intensity values of the observation target region A, to calculate an intensity value of each pixel having been adjusted. By doing so, it is possible to make the aggregate values V of intensity values of the respective observation target regions A equal to each other. By the processes of the step S3 to S5 described above, the intensity values of the multiple tomographic images D1 are standardized.

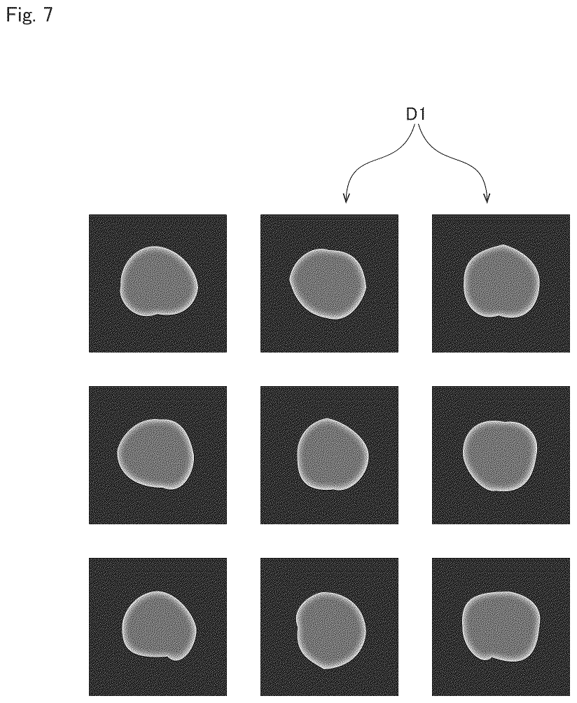

The tomographic images D1 having been subjected to standardization are stored in the storage unit 33. Further, the computer 30 displays the tomographic images D1 having been subjected to standardization on the display unit 70. FIG. 7 is a view schematically showing the multiple tomographic images D1 having been subjected to standardization. As shown in FIG. 7, in the multiple tomographic images D1 having been subjected to standardization, the observation target regions A have uniform brightness.

After that, the evaluation-value output unit 45 of the computer 30 outputs an evaluation value R of the biological sample 9 on the basis of the observation target region A having the intensity values having been adjusted in the step S5 (step S6). The evaluation value R is an indicator representing a condition of the biological sample 9. Specifically, a point-to-point distance, a tomographic area, a volume, sphericity, surface roughness, an internal hollow volume, and the like of the biological sample 9 are calculated as the evaluation value R. The calculated evaluation value R is stored in the storage unit 33. Further, the evaluation-value output unit 45 displays the calculated evaluation value R on the display unit 70. By the standardization of intensity value described above, variation in brightness among the multiple observation target regions A is reduced. Therefore, the evaluation values R can be calculated fairly for the multiple biological samples 9.

Regarding the biological samples 9 each having a three-dimensional structure, such as spheroids, variation in an amount of near infrared light reaching the inside of each biological sample 9 is liable to occur due to differences in a shape and a size among the biological samples 9. Further, in optical coherence tomography, the computer 30 assigns relative intensity values to respective coordinates on the basis of detection signals provided from the light detector 242. Thus, especially in taking an image of the biological samples 9 each having a three-dimensional structure by optical coherence tomography, there is caused a problem of easy occurrence of variation in an intensity value among the biological samples 9. However, in the observation apparatus 1 according to the present embodiment, intensity values of the multiple observation target regions A are standardized as described above, so that variation in an intensity value among the biological samples 9 can be reduced. Therefore, conditions of the internal parts of the multiple biological samples 9 can be fairly evaluated.

### <3. Modifications>

Hereinabove, one embodiment of the present invention has been described, but the present invention is not limited to the above-described embodiment.

### <3-1. First Modification>

In the above-described embodiment, an average value of multiple intensity values included in the observation target region A is calculated as the aggregate value V. However, when an average value is used, the aggregate value V is not consistent with overall brightness of the observation target region A under the influence of an outlier in a case in which there is an outlier pixel having an extremely remote intensity value in the observation target region A, in some cases. In such a case, a median value of multiple intensity values included in the observation target region A may be used as the aggregate value V. By using a median value, it is possible to reduce the influence of an outlier even in a case in which there is an outlier pixel having an extremely remote intensity value in the observation target region A, to thereby calculate the aggregate value V that reflects the overall brightness of the observation target region A.

### <3-2. Second Modification>

In the above-described embodiment, a whole of a region corresponding to the biological sample 9 in the tomographic image D1 is regarded as the observation target region A. Alternatively, a part of a region corresponding to the biological sample 9 in the tomographic image D1 may be regarded as the observation target region A.

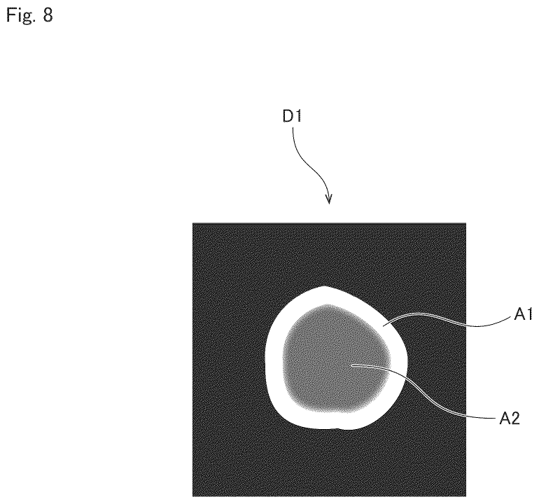

For example, in a case in which an internal part of a spheroid necroses during culture, a part near an outer surface and the internal part in the biological sample 9 have different cell structures. Further, in a case in which a spheroid is formed of a combination of multiple kinds of cells, cells of each kind are localized in some cases. In those cases, as shown in FIG. 8, multiple regions A1 and A2 having different intensity values are included in a region corresponding to the biological sample 9 in the tomographic image D1.

In such a case, the region extraction unit 42 of the computer 30 may extract one of the multiple regions A1 and A2, as the observation target region A. Specifically, in the above-described step S3, the region extraction unit 42 extracts only a region belonging to a desired intensity-value range, and thus, only a region that is desired to be evaluated among the multiple regions A1 and A2 can be regarded as the observation target region A. Alternatively, one of the multiple regions A1 and A2 may be extracted as the observation target region A with the use of deep learning.

Suppose that a whole including the regions A1 and A2 is extracted as the observation target region A in the step S3. Then, the aggregate value V calculated in the step S4 reflects average brightness of the whole including the regions A1 and A2. In this case, an intensity value to be adjusted in the step S5 is not an intensity value suitable for evaluation of only a partial region, in some cases. Thus, in a case in which it is desired to evaluate only a partial region, it is preferred to extract only the partial region as the observation target region A in the step S3.

### <3-3. Third Modification>

In the above-described embodiment, description has been given about a case in which the tomographic image D1 is subjected to standardization. Alternatively, a target of standardization may be a three-dimensional image D2. In a case in which the three-dimensional image D2 is a target of standardization, a three-dimensional region corresponding to a whole or a part of the biological sample 9 in three-dimensional coordinates forming the three-dimensional image D2 is extracted as the observation target region A in the above-described step S3. Then, in the step S4, the aggregate value V of intensity values is calculated for each of multiple observation target regions A. After that, an intensity value of each pixel in the three-dimensional observation target region A is adjusted so that a difference in the aggregate value V becomes smaller.

### <3-4. Other Modifications>

Meanwhile, in the above-described embodiment, the imaging unit 20 performs optical coherence tomography (OCT). Alternatively, the imaging unit may acquire a tomographic image or a three-dimensional image of a biological sample by other image-taking methods.

Further, in the above-described embodiment, the sample holder 90 is a well plate consisting of the multiple wells (recessed portions) 91, and each of the wells 91 holds one biological sample 9. Alternatively, one well may hold multiple biological samples. In such a case, one image may include regions respectively corresponding to the multiple biological samples. Further alternatively, the sample holder that holds a biological sample may be a dish having only one recessed portion.

Moreover, the respective elements described in the above-described embodiment and modifications may be appropriately combined unless contradiction occurs.

### REFERENCE SIGNS LIST

1 Observation apparatus
9 Biological sample
10 Stage
20 Imaging unit
21 Light source
22 Object optical system
23 Reference optical system
24 Detection unit
25 Optical fiber coupler
30 Computer
41 Image acquisition unit
42 Region extraction unit
43 Aggregate-value calculation unit
44 Intensity-value calculation unit
45 Evaluation-value output unit
70 Display unit
90 Sample holder
91 Well
A Observation target region
D1 Tomographic image
R Evaluation value
V Aggregate value

1. An image standardization method for standardizing intensity values of multiple images of a biological sample including multiple cells, comprising the steps of:
a) extracting a region corresponding to a whole or a part of the biological sample in each of the images, as an observation target region, for each of the multiple images;
b) calculating an aggregate value of intensity values for each of the multiple observation target regions; and
c) adjusting intensity values of the multiple observation target regions so that a difference in the aggregate value becomes smaller.

2. The image standardization method according to claim 1, wherein the aggregate value is an average value of multiple intensity values included in the observation target region.

3. The image standardization method according to claim 1, wherein the aggregate value is a median value of multiple intensity values included in the observation target region.

4. The image standardization method according to claim 1, wherein
the region corresponding to the biological sample in each of the images includes multiple regions having different intensity values, and
in the step a), one of the multiple regions is extracted as the observation target region.

5. The image standardization method according to claim 1, wherein the images are tomographic images or three-dimensional images of the biological sample that are acquired by optical coherence tomography.

6. An observation apparatus for observing a biological sample including multiple cells, comprising:
an image acquisition unit configured to acquire multiple images of the biological sample:
a region extraction unit configured to extract a region corresponding to a whole or a part of the biological sample in each of the images, as an observation target region, for each of the multiple images;
an aggregate-value calculation unit configured to calculate an aggregate value of intensity values, for each of the multiple observation target regions; and
an intensity-value adjustment unit configured to adjust intensity values of the multiple observation target regions so that a difference in the aggregate value becomes smaller.

7. The observation apparatus according to claim 6, wherein the aggregate value is an average value of multiple intensity values included in the observation target region.

8. The observation apparatus according to claim 6, wherein the aggregate value is a median value of multiple intensity values included in the observation target region.

9. The observation apparatus according to claim 6, wherein
the region corresponding to the biological sample in each of the images includes multiple regions having different intensity values, and
the region extraction unit extracts one of the multiple regions as the observation target region.

10. The observation apparatus according to claim 6, wherein the image acquisition unit acquires a tomographic image or a three-dimensional image of the biological sample by optical coherence tomography.

11. The observation apparatus according to claim 6, further comprising an evaluation-value output unit configured to output an evaluation value of the biological sample on the basis of an image having an intensity value having been adjusted by the intensity-value adjustment unit.
